(19) United States
(12) Patent Application Publication
(10) Pub. No.: US 2022/0026409 A1
(43) Pub. Date: Jan. 27, 2022

(54) METHOD AND SYSTEM FOR PREDICTING DISINFECTION BY-PRODUCTS IN DRINKING WATER

(71) Applicant: Jilin Jianzhu University, Jilin (CN)

(72) Inventors: Yingzi LIN, Jilin (CN); Gen LIU, Jilin (CN); Gaoqi WANG, Jilin (CN); Daihua ZHANG, Jilin (CN); Hao YANG, Jilin (CN); Yuhang WEI, Jilin (CN); Wanqing LIU, Jilin (CN)

(73) Assignee: Jilin Jianzhu University, Jilin (CN)

(21) Appl. No.: 17/075,885

(22) Filed: Oct. 21, 2020

(30) Foreign Application Priority Data
Jul. 21, 2020 (CN) 202010702802.2

(51) Int. Cl. G01N 33/18; G06N 3/08

(52) U.S. Cl. CPC G01N 33/18; G06N 3/086

(57) ABSTRACT

The disclosure provides a method and a system for predicting disinfection by-products in drinking water. The method includes: acquiring water age prediction data of the drinking water to be predicted and water quality data of the drinking water to be predicted; inputting the water age prediction data and the water quality data into an adaptive genetic BP neural network model for predicting the disinfection by-products in the drinking water to obtain prediction values of the disinfection by-products in the drinking water. The disinfection by-products in a water supply pipe network can be predicted efficiently and economically by using the method and the system for predicting the disinfection by-products in the drinking water provided by the disclosure.

### TECHNICAL FIELD

The present disclosure relates to a technical field of water quality detection, more particular to a method and a system for predicting disinfection by-products in drinking water.

### BACKGROUND

Urban water supply pipe networks are important infrastructures to ensure people's living standards, and drinking water safety attracts more and more attention. However, there are problems such as outdated facilities, incomplete information, and backward management methods in most of the current urban water supply pipe networks, resulting in “secondary pollution” for drinking water that has been treated and reached the standard. For this reason, it is necessary to maintain a proper amount of residual chlorine in the drinking water. When the chlorine-containing disinfectant is added at water plants, it will react with the organic matter in the water to generate disinfection by-products (DBPs). DBPs mainly include: trihalomethanes (THMs), haloacetic acids (HAAs), haloacetonitriles (HANs), and the like. These disinfection by-products can have great threat to human health. The low content of DBPs often cannot reach a detection limit of an existing instrument. Thus, it is required to perform pretreatment such as concentration or extraction on water samples and use instruments such as gas chromatography (GC) and gas chromatography/mass spectroscopy (GC/MS), resulting in a relatively high cost of detection, and detecting disinfection by-products consumes a large amount of time and expenditure. Therefore, a method for efficiently and economically detecting disinfection by-products in water supply pipe networks has great practical importance to ensure the safety of drinking water.

### SUMMARY OF THE INVENTION

The present disclosure intends to provide a method and a system for predicting disinfection by-products in drinking water, which can predict disinfection by-products in a water supply pipe networks efficiently and economically.

In order to achieve the above effect, the present disclosure provides the following solutions:

A method for predicting disinfection by-products in drinking water includes:

acquiring water age prediction data of the drinking water to be predicted and water quality data of the drinking water to be predicted; and

inputting the water age prediction data and the water quality data into an adaptive genetic BP neural network model for predicting the disinfection by-products in the drinking water to obtain a prediction value of the disinfection by-products in the drinking water.

Optionally, after acquiring the water age prediction data and the water quality data of the drinking water to be predicted, the method further includes:

normalizing the water age prediction data of the drinking water to be predicted and the water quality data of the drinking water to be predicted to obtain normalized water age prediction data of the drinking water to be predicted and normalized water quality data of the drinking water to be predicted.

Optionally, a specific method for generating the water age prediction data includes:

acquiring water supply pipe network parameters; wherein, the water supply pipe network parameters include: a pipe section length, a pipe diameter dimension, a pipe section flow velocity boundary condition, a flow rate of a node between pipe sections and a water head boundary condition;

establishing a hydraulic model of a water supply pipe network according to the water supply pipe network parameters; and

calculating water age of the drinking water to be predicted according to the hydraulic model of the water supply pipe network to obtain the water age prediction data.

Optionally, a specific method for constructing the adaptive genetic BP neural network model for predicting the disinfection by-products in the drinking water includes:

acquiring historical water age data, historical water quality data, and historical disinfection by-products data of the drinking water;

normalizing the historical water age data and the historical water quality data to obtain normalized historical water age data and normalized historical water quality data;

establishing a BP neural network model according to the normalized historical water age data, the normalized historical water quality data and the historical disinfection by-products data of the drinking water;

acquiring desired values of the disinfection by-products data of the drinking water; and

optimizing parameters in the BP neural network model by taking a reciprocal of a sum of squared differences between the desired values of the disinfection by-products data of the drinking water and actual values of the disinfection by-products data of the drinking water output from the BP neural network model as an objective function of an adaptive genetic algorithm, to obtain the adaptive genetic BP neural network model for predicting the disinfection by-products in the drinking water.

Optionally, establishing the BP neural network model according to the normalized historical water age data, the normalized historical water quality data and the historical disinfection by-products data of the drinking water specifically includes:

determining the number of input layer nodes of the BP neural network model according to the historical water age data and the historical water quality data;

determining the number of output layer nodes of the BP neural network model according to the historical disinfection by-products data of the drinking water;

calculating the number of hidden layer nodes of the BP neural network model according to the number of the input layer nodes and the number of the output layer nodes; and

establishing the BP neural network model according to the normalized historical water age data, the normalized historical water quality data, the historical disinfection by-products data of the drinking water, the number of the input layer nodes, the number of the output layer nodes, and the number of the hidden layer nodes.

A system for predicting disinfection by-products in drinking water, provided in the present disclosure, includes:

an acquisition module for data to be predicted, configured to acquire water age prediction data of the drinking water to be predicted and water quality data of the drinking water to be predicted; and

a prediction module for the disinfection by-products in the drinking water, configured to input the water age prediction data and the water quality data into an adaptive genetic BP neural network model for predicting the disinfection by-products in the drinking water to obtain a prediction value of the disinfection by-products in the drinking water.

Optionally, the system further includes:

a normalization module configured to normalize the water age prediction data of the drinking water to be predicted and the water quality data of the drinking water to be predicted to obtain normalized water age prediction data of the drinking water to be predicted and normalized water quality data of the drinking water to be predicted.

Optionally, the acquisition module for the data to be predicted specifically includes:

a water age prediction data generation unit configured to acquire water supply pipe network parameters, to establish a hydraulic model of a water supply pipe network by using infoworks according to the water supply pipe network parameters, and to calculate water age of the drinking water to be predicted according to the hydraulic model of the water supply pipe network to obtain the water age prediction data; wherein, the water supply pipe network parameters include: a pipe section length, a pipe diameter dimension, a pipe section flow velocity boundary condition, a flow rate of a node between pipe sections and a water head boundary condition.

Optionally, the prediction module for the drinking water disinfection by-products specifically includes:

a historical data acquisition unit, configured to acquire historical water age data, historical water quality data, and historical disinfection by-products data of the drinking water;

a historical data normalization unit, configured to normalize the historical water age data and the historical water quality data to obtain normalized historical water age data and normalized historical water quality data;

a BP neural network model establishment unit, configured to establish a BP neural network model according to the normalized historical water age data, the normalized historical water quality data and the historical disinfection by-products data of the drinking water;

an acquisition unit for desired values of the disinfection by-products data of the drinking water, configured to acquire desired values of the disinfection by-products data of the drinking water;

an establishment unit for an adaptive genetic BP neural network model for predicting the disinfection by-products in the drinking water, configured to optimize parameters in the BP neural network model by taking a reciprocal of a sum of squared differences between the desired values of the disinfection by-products data of the drinking water and actual values of the disinfection by-products data of the drinking water output from the BP neural network model as an objective function of an adaptive genetic algorithm, to obtain the adaptive genetic BP neural network model for predicting the disinfection by-products in the drinking water.

Optionally, the BP neural network model establishment unit specifically includes:

a determination subunit for a number of input layer nodes, configured to determine the number of the input layer nodes of the BP neural network model according to the historical water age data and the historical water quality data;

a determination subunit for a number of output layer nodes, configured to determine the number of the output layer nodes of the BP neural network model according to the historical disinfection by-products data of the drinking water;

a determination subunit for a number of hidden layer nodes, configured to calculate the number of the hidden layer nodes of the BP neural network model according to the number of the input layer nodes and the number of the output layer nodes; and

an establishment subunit for the BP neural network model, configured to establish the BP neural network model according to the normalized historical water age data, the normalized historical water quality data, the historical disinfection by-products data of the drinking water, the number of the input layer nodes, the number of the output layer nodes, and the number of the hidden layer nodes.

Compared with the conventional technology, the beneficial effects of the present disclosure are as follows:

the present disclosure provides a method and a system for predicting disinfection by-products in drinking water. In the present disclosure, the prediction value of the disinfection by-products in the drinking water is obtained by inputting the water age prediction data of the drinking water to be predicted and the water quality data of the drinking water to be predicted into the adaptive genetic BP neural network model for predicting the disinfection by-products in the drinking water, which can achieve replacing a detection of the disinfection by-products with a detection of conventional water quality indicators and a purpose of timely discovering the disinfection by-products and reducing detection costs. At the same time, the adaptive genetic BP neural network model adopted in the present disclosure has fast convergence speed and small prediction error.

### BRIEF DESCRIPTION OF THE DRAWINGS

In order to more clearly illustrate embodiments of the present disclosure or technical solutions in the conventional technology, accompanying drawings used in the embodiments will now be described briefly. It is obvious that the drawings in the following description are only some embodiments of the present disclosure, and that those skilled in the art can obtain other drawings from these drawings without involving any inventive effort.

FIG. 1 is a flow chart of a method for predicting disinfection by-products in drinking water in an embodiment of the present disclosure; and

FIG. 2 is a flow chart of a system for predicting disinfection by-products in drinking water in an embodiment of the present disclosure.

### DETAILED DESCRIPTION OF THE EMBODIMENTS

In the following, technical solutions in the embodiments of the present disclosure will be clearly and completely described with reference to the drawings in the embodiments of the present disclosure. Obviously, the described embodiments are only a part of the embodiments of the present disclosure, but not all the embodiments. Based on the embodiments of the present disclosure, all other embodiments obtained by a person of ordinary skill in the art without involving any inventive effort are within the scope of the present disclosure.

The present disclosure intends to provide a method and a system for predicting disinfection by-products in drinking water, which can predict disinfection by-products in a water supply pipe network efficiently and economically.

To further clarify the above objects, features and advantages of the present disclosure, a more particular description of the disclosure will be rendered by reference to the accompanying drawings and specific embodiments thereof.

### Embodiment

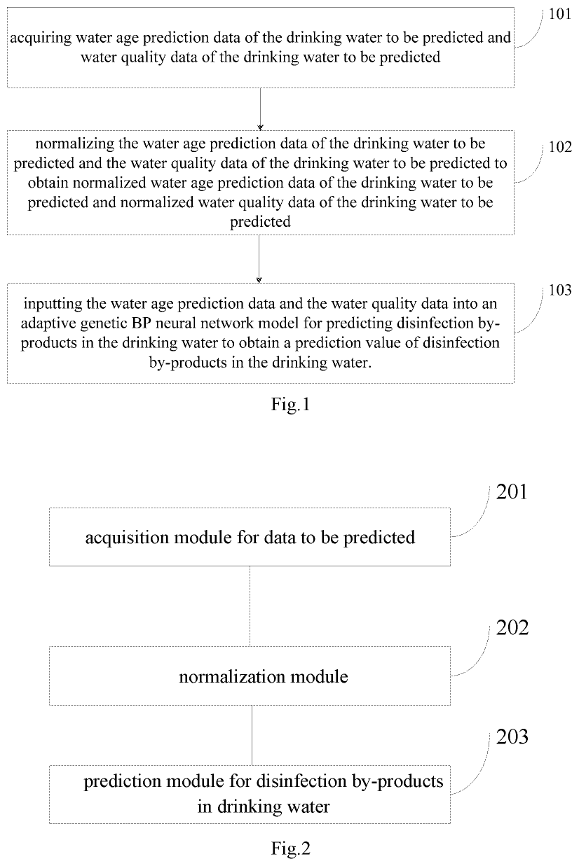

FIG. 1 is a flow chart of a method for predicting disinfection by-products in drinking water in an embodiment of the present disclosure. As shown in FIG. 1, a method for predicting disinfection by-products in drinking water includes steps 101-103 as follows.

Step 101: water age prediction data (Tᵢ) of the drinking water to be predicted and water quality data of the drinking water to be predicted are acquired. The water quality data includes: residual chlorine (Cl₂), turbidity (NTU), potential of hydrogen (PH), ammonia nitrogen (NH₃—N), nitrate nitrogen (NO₃⁻—N), nitrite nitrogen (NO₂⁻—N), total organic carbon (TOC), ultraviolet absorbance (UV₂₅₄), fluoride ion (F⁻), and total iron (Fe).

A specific method for generating the water age prediction data includes: acquiring water supply pipe network parameters including a pipe section length, a pipe diameter dimension, a pipe section flow velocity boundary condition, a flow rate of a node between pipe sections and a water head boundary condition; establishing a hydraulic model of a water supply pipe network by using infoworks according to the water supply pipe network parameters; predicting the water age of the drinking water to be predicted according to the hydraulic model of the water supply pipe network to obtain the water age prediction data.

Specifically,

An alignment layout of an urban water supply pipe network that needs to be rebuilt and expanded involves: pipe section N (N=1, 2, 3, 4 . . . ), node number n (n=1, 2, 3, 4 . . . ), pipe section length (Lᵢⱼ, i is an upstream node of the pipe section, and j is a downstream node of the pipe section), a standard pipe diameter list (Dᵢⱼ, i is the upstream node of the pipe section, and j is the downstream node of the pipe section) and a unit length cost table, a flow velocity boundary condition of the pipe section (Vᵢⱼ, i is the upstream node of the pipe section, and j is the downstream node of the pipe section), a flow rate of a node (Qᵢⱼ, i is the upstream node of the pipe section, and j is the downstream node of the pipe section) and a water head boundary condition.

A hydraulic model of a water supply pipe network is established by using an infoworks software, and the flow rate and a pressure are checked, and then a water age dynamic model is established in the water quality part to obtain the water age data Tn (n is the node number). The model is established by importing CAD drawings into infoworks, and then the water supply pipe network parameters are input into a network topology diagram to be checked to determine whether the pressure and the flow rate are within reasonable ranges.

Step 102: The water age prediction data of the drinking water to be predicted and the water quality data of the drinking water to be predicted are normalized to obtain normalized water age prediction data of the drinking water to be predicted and normalized water quality data of the drinking water to be predicted.

Since different water quality indicators have different contents in the pipe network and different units, in order to avoid influences on model accuracy due to a difference in the order of magnitude between the indicators, it is necessary to normalize the water quality indicators. Since the numerical value of the water quality indicator is certainly greater than 0, the water quality indicator needs to be normalized to be within a range of [0, 1], and a normalization formula is as follows:

$$\hat{X} = \frac{X - {X}_{min}}{{X}_{max} - {X}_{min}}$$

Wherein, \hat{X} is a numerical value of a normalized water quality indicator; X is a value of current water quality data; Xₘₐₓ is the maximum value of an original water quality data sequence; Xₘᵢₙ is the minimum value of the original water quality data sequence.

Step 103: The water age prediction data and the water quality data are input into an adaptive genetic BP neural network model for predicting disinfection by-products in the drinking water to obtain a prediction value of the disinfection by-products in the drinking water. The disinfection by-products in the drinking water are trihalomethanes and haloacetonitriles.

A specific method for constructing the adaptive genetic BP neural network model for predicting disinfection by-products in the drinking water includes:

1) Acquiring historical water age data, historical water quality data, and historical disinfection by-products data of the drinking water;

2) Normalizing the historical water age data and the historical water quality data to obtain normalized historical water age data and normalized historical water quality data;

3) Establishing a BP neural network model according to the normalized historical water age data, the normalized historical water quality data and the historical disinfection by-products data of the drinking water, wherein the step of establishing the BP neural network model specifically include:

determining the number of the input layer nodes of the BP neural network model according to the historical water age data and the historical water quality data;

determining the number of the output layer nodes of the BP neural network model according to the historical disinfection by-products data of the drinking water;

calculating the number of the hidden layer nodes of the BP neural network model according to the number of the input layer nodes and the number of the output layer nodes; establishing the BP neural network model according to the normalized historical water age data, the normalized historical water quality data, the historical disinfection by-products data of the drinking water, the number of the input layer nodes, the number of the output layer nodes, and the number of the hidden layer nodes;

4) Acquiring desired values of the disinfection by-products data of the drinking water; and

5) Optimizing parameters in the BP neural network model by taking a reciprocal of a sum of squared differences between the desired values of the disinfection by-products data of the drinking water and actual values of the disinfection by-products data of the drinking water output from the BP neural network model as an objective function of an adaptive genetic algorithm, to obtain the adaptive genetic BP neural network model for predicting disinfection by-products in the drinking water.

Specifically,

for the BP neural network, the BP neural network includes three network layers of an input layer, a hidden layer and an output layer, and the number of hidden layer neurons is determined according to the following formula:

h=\sqrt{m+n}+α

wherein, h is the number of the hidden layer nodes, m is the number of the input layer nodes, n is the number of the output layer nodes, and a is an adjustment constant between 1 and 10.

According to input layer node data, it can be seen that the number of the hidden layer neurons is between 4 and 14, and then the hidden layer nodes are increased from 4 to 14 with cross-validation. The learning rate is gradually increased from 0.1 to 0.8 to obtain a training error. Generally, a random number between −1 and 1 is taken as an initial weight, and a selection range of a training objective error is set to be 1.0×10⁻³-1.0×10⁻⁵.

For the adaptive genetic algorithm, the population scale is selected between 100 and 350 depending on an actual situation. Herein, the optimized mean absolute percentage error (MAPE) for different population sizes are analyzed respectively by using real number coding.

For an adaptation function, in a three-layer BP network (the number of the input layer nodes is M, the number of the hidden layer nodes is N, and the number of the output layer nodes is T), the result of the output layer and the input value of the input layer can be expressed by the following derivation:

Input of the i^{th} node in the hidden layer:

netᵢ=Σᵢ₌₁^{N}wᵢⱼpᵢ+θᵢ.

Output of the i^{th} node in the hidden layer:

Oᵢ=Ø(netᵢ)=Ø(Σᵢ₌₁^{N}wᵢⱼpᵢ+θᵢ).

Input of the j^{th} node in the output layer:

netⱼ=Σⱼ₌₁^{T}vᵢⱼOᵢ=Σⱼ₌₁^{T}vᵢⱼØ(Σᵢ₌₁^{N}wᵢⱼpᵢ+θᵢ).

Output of the j^{th} node in the output layer:

Oⱼ=ψ(netⱼ)=ψ(Σⱼ₌₁^{T}vᵢⱼOᵢ)=ψ(Σⱼ₌₁^{T}vᵢⱼØ(Σᵢ₌₁^{N}wᵢⱼpᵢ+θᵢ)+γᵢ).

wherein: pᵢ is an input of the i^{th} node in the input layer; Oⱼ is an output of the j^{th} node in the output layer; wᵢⱼ is a weight from the i^{th} node in the output layer to the j^{th} node in the hidden layer; vᵢⱼ is a weight from the i^{th} node in the hidden layer to the j^{th} node in the output layer; θᵢ is a threshold of the i^{th} node in the hidden layer; γᵢ is a threshold of the i^{th} node in the output layer; Ø is an activation function of the hidden layer; Ψ is an activation function of the output layer;

The total error of the network is ε, and then the error function is:

Eₚ=½Σᵢ₌₁^{M}Σⱼ₌₁^{N}(T^{k}−Oₖ)².

The objective function of the genetic algorithm is carried out in an increasing direction of a fitness function, so herein a reciprocal of a sum of squared errors is taken as the fitness function, and the fitness function is set as follows:

$$F ( w , v , θ , γ ) = \frac{1}{{\sum }_{i = 1}^{M} {\sum }_{j = 1}^{N} {( {T}_{k} - {O}_{k} )}^{2}}$$

wherein: Tₖ is the desired output; Oₖ is the actual output.

With an increase in the number of literations, the genetic algorithm more and more approaches the optimized objective value, and generally, the number of literations of the genetic algorithm is set to be 500.

The embodiments provided in the present disclosure uses a cross-validation to select the optimal parameters. After experiments, an average error percentage is the smallest when a population size is 100, a genetic algebra is 100, the number of hidden layer neurons is 11, a learning efficiency is 0.1, an objective error is 10⁻⁴, and the number of trainings is 2000.

FIG. 2 is a structural drawing of a system for predicting disinfection by-products in drinking water in an embodiment of the present disclosure. As shown in FIG. 2, the system for predicting the disinfection by-products in the drinking water includes an acquisition module 201 for data to be predicted, a normalization module 202, and a prediction module 203 for disinfection by-products in drinking water.

The acquisition module 201 for the data to be predicted is configured to acquire water age prediction data of the drinking water to be predicted and water quality data of the drinking water to be predicted.

The acquisition module 201 for the data to be predicted specifically includes:

a generation unit for water age prediction data configured to acquire water supply pipe network parameters, to establish a hydraulic model of a water supply pipe network by using infoworks according to the water supply pipe network parameters, and to predict the water age of the drinking water to be predicted according to the hydraulic model of the water supply pipe network to obtain the water age prediction data; wherein, the water supply pipe network parameters include: a pipe section length, a pipe diameter dimension, a pipe section flow velocity boundary condition, a flow rate of a node between pipe sections and a water head boundary condition.

The normalization module 202 is configured to normalize the water age prediction data of the drinking water to be predicted and the water quality data of the drinking water to be predicted to obtain normalized water age prediction data of the drinking water to be predicted and normalized water quality data of the drinking water to be predicted.

The prediction module 203 for the disinfection by-products in the drinking water is configured to input the water age prediction data and the water quality data into an adaptive genetic BP neural network model for predicting the disinfection by-products in the drinking water to obtain a prediction value of disinfection by-products in the drinking water.

The prediction module 203 for the disinfection by-products in the drinking water specifically includes:

a historical data acquisition unit configured to acquire historical water age data, historical water quality data, and historical disinfection by-products data of the drinking water;

a historical data normalization unit configured to normalize the historical water age data and the historical water quality data to obtain normalized historical water age data and normalized historical water quality data; and

a BP neural network model establishment unit configured to establish a BP neural network model according to the normalized historical water age data, the normalized historical water quality data and the historical disinfection by-products data of the drinking water.

The BP neural network model establishment unit specifically includes:

a determination subunit for the number of input layer nodes configured to determine the number of the input layer nodes of the BP neural network model according to the historical water age data and the historical water quality data;

a determination subunit for the number of the output layer nodes configured to determine the number of the output layer nodes of the BP neural network model according to the historical disinfection by-products data of the drinking water;

a determination subunit for the number of the hidden layer nodes configured to calculate the number of the hidden layer nodes of the BP neural network model according to the number of the input layer nodes and the number of the output layer nodes;

an establishment subunit for the BP neural network model configured to establish the BP neural network model according to the normalized historical water age data, the normalized historical water quality data, the historical disinfection by-products data of the drinking water, the number of the input layer nodes, the number of the output layer nodes, and the number of the hidden layer nodes;

a acquisition unit for desired values of the disinfection by-products data of the drinking water configured to acquire the desired values of the disinfection by-products data of the drinking water; and

An establishment unit for an adaptive genetic BP neural network model for predicting the disinfection by-products in the drinking water configured to optimize parameters in the BP neural network model by taking a reciprocal of a sum of squared differences between the desired values of the disinfection by-products data of the drinking water and actual values of the disinfection by-products data of the drinking water output from the BP neural network model as an objective function of an adaptive genetic algorithm, to obtain the adaptive genetic BP neural network model for predicting the disinfection by-products in the drinking water.

The system disclosed by the embodiment corresponds to the method disclosed by the embodiment and thus is briefly described, and the relevant parts can refer to the portion of the method.

The principles and implementation of the present disclosure have been described herein with specific examples, and the above embodiments are presented to aid in the understanding of the methods and core concepts of the present disclosure; meanwhile, those skilled in the art may make some changes in both the detailed description and an application scope according to the teachings of this disclosure. In conclusion, the contents of the description should not be construed as limiting the disclosure.

What is claimed:

1. A method for predicting disinfection by-products in drinking water, comprising:
acquiring water age prediction data of the drinking water to be predicted and water quality data of the drinking water to be predicted; and
inputting the water age prediction data and the water quality data into an adaptive genetic BP neural network model for predicting the disinfection by-products in the drinking water to obtain a prediction value of the disinfection by-products in the drinking water.

2. The method for predicting the disinfection by-products in the drinking water according to claim 1, wherein, after acquiring the water age prediction data and the water quality data of the drinking water to be predicted, the method further comprises:
normalizing the water age prediction data of the drinking water to be predicted and the water quality data of the drinking water to be predicted to obtain normalized water age prediction data of the drinking water to be predicted and normalized water quality data of the drinking water to be predicted.

3. The method for predicting the disinfection by-products in the drinking water according to claim 2, wherein a method for generating the water age prediction data comprises:
acquiring water supply pipe network parameters; wherein, the water supply pipe network parameters comprise: a pipe section length, a pipe diameter dimension, a pipe section flow velocity boundary condition, a flow rate of a node between pipe sections and a water head boundary condition;
establishing a hydraulic model of a water supply pipe network according to the water supply pipe network parameters; and
calculating water age of the drinking water to be predicted according to the hydraulic model of the water supply pipe network to obtain the water age prediction data.

4. The method for predicting the disinfection by-products in the drinking water according to claim 3, wherein a method for constructing the adaptive genetic BP neural network model for predicting the disinfection by-products in the drinking water comprises:
acquiring historical water age data, historical water quality data, and historical disinfection by-products data of the drinking water;
normalizing the historical water age data and the historical water quality data to obtain normalized historical water age data and normalized historical water quality data;
establishing a BP neural network model according to the normalized historical water age data, the normalized historical water quality data and the historical disinfection by-products data of the drinking water;
acquiring desired values of the disinfection by-products data of the drinking water; and
optimizing parameters in the BP neural network model by taking a reciprocal of a sum of squared differences between the desired values of the disinfection by-products data of the drinking water and actual values of the disinfection by-products data of the drinking water output from the BP neural network model as an objective function of an adaptive genetic algorithm, to obtain the adaptive genetic BP neural network model for predicting the disinfection by-products in the drinking water.

5. The method for predicting the disinfection by-products in the drinking water according to claim 4, wherein establishing the BP neural network model according to the normalized historical water age data, the normalized historical water quality data and the historical disinfection by-products data of the drinking water comprises:
determining a number of input layer nodes of the BP neural network model according to the historical water age data and the historical water quality data;
determining a number of output layer nodes of the BP neural network model according to the historical disinfection by-products data of the drinking water;
calculating a number of hidden layer nodes of the BP neural network model according to the number of the input layer nodes and the number of the output layer nodes; and
establishing the BP neural network model according to the normalized historical water age data, the normalized historical water quality data, the historical disinfection by-products data of the drinking water, the number of the input layer nodes, the number of the output layer nodes, and the number of the hidden layer nodes.

6. A system for predicting disinfection by-products in drinking water, comprising:
an acquisition module for data to be predicted, configured to acquire water age prediction data of the drinking water to be predicted and water quality data of the drinking water to be predicted; and
a prediction module for the disinfection by-products in the drinking water, configured to input the water age prediction data and the water quality data into an adaptive genetic BP neural network model for predicting the disinfection by-products in the drinking water to obtain a prediction value of the disinfection by-products in the drinking water.

7. The system for predicting the disinfection by-products in the drinking water according to claim 6, wherein the system further comprises:
a normalization module configured to normalize the water age prediction data of the drinking water to be predicted and the water quality data of the drinking water to be predicted to obtain normalized water age prediction data of the drinking water to be predicted and normalized water quality data of the drinking water to be predicted.

8. The system for predicting the disinfection by-products in the drinking water according to claim 7, wherein the acquisition module for the data to be predicted comprises:
a water age prediction data generation unit configured to acquire water supply pipe network parameters, to establish a hydraulic model of a water supply pipe network by using infoworks according to the water supply pipe network parameters, and to calculate water age of the drinking water to be predicted according to the hydraulic model of the water supply pipe network to obtain the water age prediction data; wherein, the water supply pipe network parameters comprise: a pipe section length, a pipe diameter dimension, a pipe section flow velocity boundary condition, a flow rate of a node between pipe sections and a water head boundary condition.

9. The system for predicting the disinfection by-products in the drinking water according to claim 8, wherein the prediction module for the disinfection by-products in the drinking water comprises:
a historical data acquisition unit, configured to acquire historical water age data, historical water quality data, and historical disinfection by-products data of the drinking water;
a historical data normalization unit, configured to normalize the historical water age data and the historical water quality data to obtain normalized historical water age data and normalized historical water quality data;
a BP neural network model establishment unit, configured to establish a BP neural network model according to the normalized historical water age data, the normalized historical water quality data and the historical disinfection by-products data of the drinking water;
an acquisition unit for desired values of the disinfection by-products data of the drinking water, configured to acquire the desired values of the disinfection by-products data of the drinking water; and
an establishment unit for an adaptive genetic BP neural network model for predicting the disinfection by-products in the drinking water, configured to optimize parameters in the BP neural network model by taking a reciprocal of a sum of squared differences between the desired values of the disinfection by-products data of the drinking water and actual values of the disinfection by-products data of the drinking water output from the BP neural network model as an objective function of an adaptive genetic algorithm, to obtain the adaptive genetic BP neural network model for predicting the disinfection by-products in the drinking water.

10. The system for predicting the disinfection by-products in the drinking water according to claim 9, wherein the BP neural network model establishment unit comprises:
a determination subunit for a number of input layer nodes, configured to determine the number of the input layer nodes of the BP neural network model according to the historical water age data and the historical water quality data;
a determination subunit for a number of output layer nodes, configured to determine the number of output layer nodes of the BP neural network model according to the historical disinfection by-products data of the drinking water;
a determination subunit for a number of hidden layer nodes, configured to calculate the number of the hidden layer nodes of the BP neural network model according to the number of the input layer nodes and the number of the output layer nodes; and
an establishment subunit for the BP neural network model, configured to establish the BP neural network model according to the normalized historical water age data, the normalized historical water quality data, the historical disinfection by-products data of the drinking water, the number of the input layer nodes, the number of the output layer nodes, and the number of the hidden layer nodes.
